(19) United States
(12) Patent Application Publication
(10) Pub. No.: US 2008/0267358 A1
(43) Pub. Date: Oct. 30, 2008

(54) PATIENT COUCH APPARATUS, DIAGNOSTIC IMAGING APPARATUS, AND METHOD OF OPERATING PATIENT COUCH APPARATUS

(76) Inventor: Yoichi Hiyama, Sakura-shi (JP)

Correspondence Address: NIXON & VANDERHYE, PC, 901 NORTH GLEBE ROAD, 11TH FLOOR, ARLINGTON, VA, 22203, US

(21) Appl. No.: 12/107,142

(22) Filed: Apr. 22, 2008

(30) Foreign Application Priority Data
Apr. 27, 2007 (JP) 2007-119489
Mar. 3, 2008 (JP) 2008-052292

(51) Int. Cl. A61B 6/04

(52) U.S. Cl. 378/209

(57) ABSTRACT

A patient couch apparatus includes a top plate on which a subject is laid, a top plate frame portion that supports the top plate such that the top plate is movable into an imaging region in a cavity of a gantry, and an up-down-lifting frame portion that is allowed to move to up-down directions by a V-shaped link and a cylinder. The V-shaped link and the cylinder are located closer to the gantry than a rotation axis of the top plate frame portion.

### CROSS-REFERENCE TO RELATED APPLICATIONS

This application is based upon and claims the benefit of priority from the prior Japanese Patent Application No. 2007-119489, filed on Apr. 27, 2007 and No. 2008-52292, filed on Mar. 3, 2008; the entire contents of which are incorporated herein by reference.

### BACKGROUND OF THE INVENTION

1. Field of the Invention

The present invention relates to a patient couch apparatus for use in a diagnostic imaging apparatus, a diagnostic imaging apparatus, and a method of operating a patient couch apparatus.

2. Description of the Related Art

Conventional diagnostic imaging apparatuses, such as a magnetic resonance imaging (MRI) apparatus and an X-ray computed tomography (CT) apparatus, generally include a gantry and a patient couch apparatus on which a subject is laid. The gantry has a cavity, into which a subject is inserted, and an imaging unit that performs imaging of the subject positioned in an imaging region inside the cavity.

FIG. 24 depicts a conventional diagnostic imaging apparatus. As shown in FIG. 24, a patient couch apparatus for use in a diagnostic imaging apparatus is provided in front of a cavity in a gantry, and includes a top plate on which a subject is laid. The patient couch apparatus moves the top plate in a longitudinal direction of the top plate, thereby moving the subject into or out of an imaging space inside the gantry (see, for example, JP-A 2006-129938 (KOKAI), or JP-A H2-55042 (KOKAI)).

While the diagnostic imaging apparatus is generally located in a special imaging room in a department of radiology or a like, in some instances, a layout of the imaging room undesirably restricts a direction, along which a subject approaches the patient couch apparatus. FIG. 25 is a schematic diagram for explaining the inconvenience pertaining to the conventional diagnostic imaging apparatus. An example layout that restricts a direction along which a subject approaches the patient couch apparatus to a lateral direction; that is, a direction perpendicular to the patient couch apparatus as shown in FIG. 25, will be described. To transfer a subject carried on a stretcher onto to a top plate, it is required to change an orientation of a stretcher so that the stretcher is aligned parallel to the top plate.

However, a narrow imaging room, a drip being given to the subject on the stretcher, or other condition makes it difficult to change the orientation of the stretcher. Transferring the subject without changing the orientation of the stretcher is not only difficult but also disadvantageous in that such transferring can put a heavy load on a patient; i.e., the subject.

Further, a patient couch apparatus for use in a diagnostic imaging apparatus generally has, below the top plate, an up-down lifting mechanism that moves the top plate up and down. This arrangement causes an operator to fail to put the operator's foot under the top plate to secure an enough foothold, thereby preventing the operator from taking a stable posture. Consequently, the operator is required to transfer a subject in an unstable posture.

Because the subject is generally a sick or injured patient, the operator is required to transfer the subject with a greatest possible care. Hence, facilitating transfer of a subject to a patient couch apparatus even when a direction, along which the subject approaches the patient couch apparatus, is limited is in great demand.

### SUMMARY OF THE INVENTION

According to one aspect of the present invention, a patient couch apparatus includes a top plate on which a subject is laid; and a frame portion that is provided on a gantry that performs imaging the subject, movable into an imaging region inside the gantry, and rotatably supports the top plate in an area outside the imaging region.

According to another aspect of the present invention, a patient couch apparatus includes a top plate on which a subject is laid; an up-down lifting mechanism that is provided on a gantry that performs imaging the subject, and supports the top plate movably to up-down directions.

According to still another aspect of the present invention, a diagnostic imaging apparatus includes a gantry that performs imaging a subject; and a patient couch apparatus on which the subject is laid, wherein the patient couch apparatus includes a top plate on which the subject is laid; and a frame portion that is provided on the gantry, movable into an imaging region inside the gantry, and rotatably supports the top plate in an area outside the imaging region.

According to still another aspect of the present invention, a diagnostic imaging apparatus includes a gantry that performs imaging a subject; and a patient couch apparatus on which the subject is laid, wherein the patient couch apparatus includes a top plate on which the subject is laid; and an up-down lifting mechanism that is provided on the gantry and supports the top plate movably to up-down directions.

According to still another aspect of the present invention, a method of operating a patient couch apparatus includes rotating a top plate frame portion that supports a top plate on which a subject is laid, to a predetermined subject-lying position at which the subject is laid on the top plate; rotating the top plate frame portion from the subject-lying position to a predetermined top plate reference position at which the top plate is perpendicular to a front plane of a cavity of a gantry that performs imaging the subject; moving the top plate frame portion from the top plate reference position to an inside-top plate standby position of a height at which the top plate is allowed to be inserted into the cavity of the gantry, by using an up-down lifting mechanism that is provided on the gantry and supports the top plate frame portion movably to up-down directions; and moving the top plate from the inside-top plate standby position to a predetermined imaging position inside the gantry.

### BRIEF DESCRIPTION OF THE DRAWINGS

FIGS. 1A and 1B are schematic diagrams for explaining an MRI apparatus according to a first embodiment of the present invention;

FIGS. 2A, 2B, and 2C are external views of an overall configuration of the MRI apparatus according to the first embodiment;

FIGS. 3A, 3B, and 3C are schematic diagrams illustrating an internal configuration of the MRI apparatus according to the first embodiment;

FIG. 4 is a diagram illustrating a top plate frame portion in a rotated position;

FIG. 5 is a diagram illustrating a rotating mechanism provided on the top plate frame portion and an upper support member;

FIG. 6 is a diagram illustrating RF coil connectors provided on a patient couch apparatus according the first embodiment;

FIG. 7 is a flowchart of a sequence of operations performed by the patient couch apparatus according to the first embodiment for imaging;

FIGS. 8A, 8B, and 8C are external views of an overall configuration of an MRI apparatus according to a second embodiment of the present invention;

FIGS. 9A, 9B, and 9C are schematic diagrams illustrating an internal configuration of the MRI apparatus according to the second embodiment;

FIGS. 10A and 10B are diagrams illustrating an up-down lifting mechanism of an up-down-lifting frame unit according to the second embodiment;

FIG. 11 is a first diagram illustrating a transfer of the top plate from a stretcher to a patient couch apparatus of an MRI apparatus according to a third embodiment of the present invention;

FIG. 12 is a second diagram illustrating a transfer of the top plate from a stretcher to a patient couch apparatus of an MRI apparatus according to a third embodiment of the present invention;

FIG. 13 is a third diagram illustrating a transfer of the top plate from a stretcher to a patient couch apparatus of an MRI apparatus according to a third embodiment of the present invention;

FIG. 14 is a fourth diagram illustrating a transfer of the top plate from a stretcher to a patient couch apparatus of an MRI apparatus according to a third embodiment of the present invention;

FIG. 15 is a fifth diagram illustrating a transfer of the top plate from a stretcher to a patient couch apparatus of an MRI apparatus according to a third embodiment of the present invention;

FIG. 16 is a first diagram illustrating a transfer of the top plate from the patient couch apparatus to the stretcher of the MRI apparatus according to the third embodiment;

FIG. 17 is a second diagram illustrating a transfer of the top plate from the patient couch apparatus to the stretcher of the MRI apparatus according to the third embodiment;

FIG. 18 is a third diagram illustrating a transfer of the top plate from the patient couch apparatus to the stretcher of the MRI apparatus according to the third embodiment;

FIG. 19 is a fourth diagram illustrating a transfer of the top plate from the patient couch apparatus to the stretcher of the MRI apparatus according to the third embodiment;

FIG. 20 is a fifth diagram illustrating a transfer of the top plate from the patient couch apparatus to the stretcher of the MRI apparatus according to the third embodiment;

FIG. 21 is a sixth diagram illustrating a transfer of the top plate from the patient couch apparatus to the stretcher of the MRI apparatus according to the third embodiment;

FIG. 22 is a first diagram illustrating a transfer of the top plate of the MRI apparatus according to the third embodiment, the transfer being effected without rotating the top plate frame portion;

FIG. 23 is a second diagram illustrating a transfer of the top plate of the MRI apparatus according to the third embodiment, the transfer being effected without rotating the top plate frame portion;

FIG. 24 is a diagram illustrating a conventional diagnostic imaging apparatus; and

FIG. 25 is a schematic diagram for explaining a problem pertaining to the conventional diagnostic imaging apparatus.

### DETAILED DESCRIPTION OF THE INVENTION

Exemplary embodiments of a patient couch apparatus, a diagnostic imaging apparatus, and a method of operating a patient couch apparatus according to the present invention will be explained below in detail with reference to the accompanying drawings. The embodiments below will describe examples, in which the present invention is applied to an MRI apparatus including a patient couch apparatus.

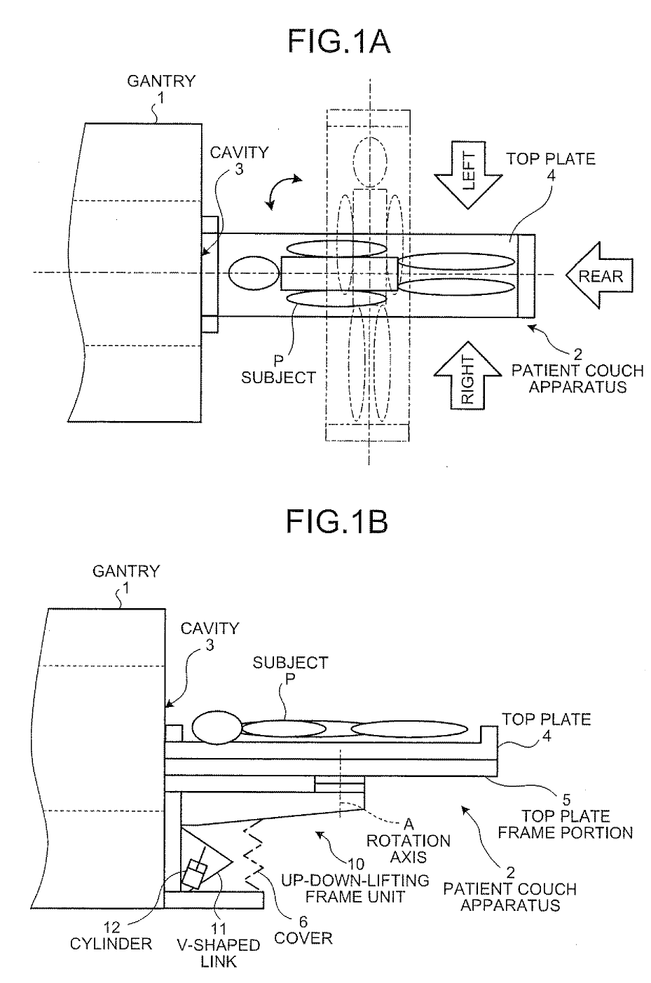

An overview of an MRI apparatus according to a first embodiment of the present invention will be described first. FIGS. 1A and 1B are schematic diagrams for explaining the MRI apparatus according to the first embodiment. As shown in FIG. 1A, the MRI apparatus includes a gantry 1 and a patient couch apparatus 2.

The gantry 1 has a cavity 3 to receive a subject P inserted thereinto. The gantry 1 generates a static magnetic field and gradient magnetic fields in an imaging region in the cavity 3, thereby irradiating radio frequency (RF) pulses to the subject P placed in the generated magnetic field. In response to the RF pulses, nuclear magnetic resonance (NMR) signals are emitted from the subject P. The NMR signals emitted from the subject P are detected by, e.g., an RF coil attached to the subject P, and sent to an image processor (not shown), such as a computer. The image processor performs predetermined computation based on the thus-sent signals, and reconstructs an image of the subject P.

The patient couch apparatus 2 is located outside the gantry 1. As shown in FIG. 1B, the patient couch apparatus 2 includes a top plate 4 on which the subject P is laid, a top plate frame portion 5 that supports the top plate 4 from below, and an up-down-lifting frame unit 10 that supports the top plate frame portion 5 from below.

The top plate frame portion 5 supports the top plate 4 such that the top plate 4 is movable into the imaging region in the cavity 3 of the gantry 1. The up-down-lifting frame unit 10 supports the top plate frame portion 5 such that the top plate frame portion 5 is rotatable about a rotation axis A, and has a V-shaped link 11 and a cylinder 12 inside a cover 6. The V-shaped link 11 and the cylinder 12 are located closer to the gantry 1 than the rotation axis A of the top plate frame portion 5. The up-down-lifting frame unit 10 vertically movably supports the top plate frame portion 5 with the V-shaped link 11 and the cylinder 12.

More specifically, in the MRI apparatus according to the first embodiment, because the top plate frame portion 5 is rotatable, the top plate 4 can be rotated to a position, at which transfer of the subject P is easily effected even when changing the orientation of the stretcher is prevented. Furthermore, in the MRI apparatus according to the first embodiment, the V-shaped link 11 and the cylinder 12, which move the top plate frame portion 5 up and down, are located closer to the gantry 1 than the rotation axis A of the top plate frame portion 5. Accordingly, a space for a foothold is secured under the top plate 4.

As described above, the MRI apparatus according to the first embodiment allows to lay the subject P on the top plate 4 easily even when a direction, along which the subject P approaches the patient couch apparatus 2, is limited.

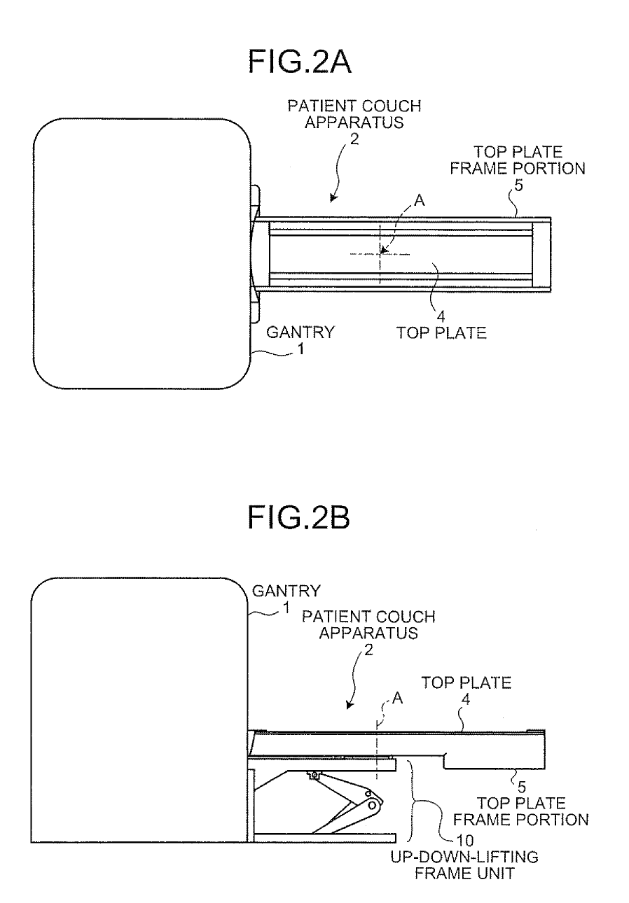

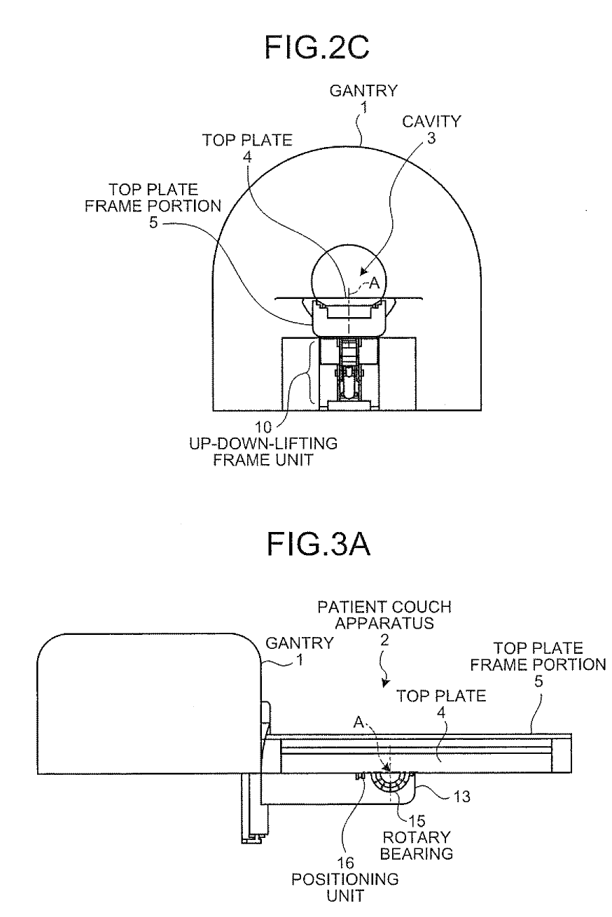

The overall configuration of the MRI apparatus according to the first embodiment will be described below. FIGS. 2A, 2B, and 2C are external views of the overall configuration of the MRI apparatus according to the first embodiment, in which FIG. 2A is a top view, FIG. 2B is a side view, and FIG. 2C is a front view. As shown in FIGS. 2A, 2B, and 2C, the MRI apparatus includes the gantry 1 and the patient couch apparatus 2.

The gantry 1 has the cavity 3 to receive the subject P inserted thereinto. The gantry 1 also has a static-magnetic-field magnet (not shown) that generates a static magnetic field in the imaging region in the cavity 3, gradient-magnetic-field coils (not shown) that generate gradient magnetic fields, and an RF transmitter (not shown) that irradiates RF pulses to the subject P placed in the static magnetic field generated by the static-magnetic-field magnet.

By irradiating the RF pulse to the subject P inside the gantry 1, the subject P emits NMR signals. The NMR signals emitted from the subject P are detected by, e.g., the RF coil attached to the subject P, and sent to the image processor (not shown), such as a computer. The image processor performs predetermined computation based on the thus-sent signals and reconstructs an image of the subject P.

The patient couch apparatus 2 is located outside the gantry 1 and includes the top plate 4 on which the subject P is laid, the top plate frame portion 5, and the up-down-lifting frame unit 10.

The top plate frame portion 5 is configured to support the top plate 4 such that the top plate 4 is movable into the imaging region in the cavity 3 of the gantry 1. More specifically, the top plate frame portion 5 supports the top plate 4 from below and internally incorporates a motor (not shown) and the like. In accordance with an instruction supplied from an operator, the motor is configured to be able to move the top plate 4 in a longitudinal direction of the top plate 4 between an inside-top plate standby position and a predetermined imaging position inside the cavity 3 of the gantry 1. The inside-top plate standby position is a position of a height at which the top plate 4 is allowed to be inserted into the cavity 3 of the gantry 1.

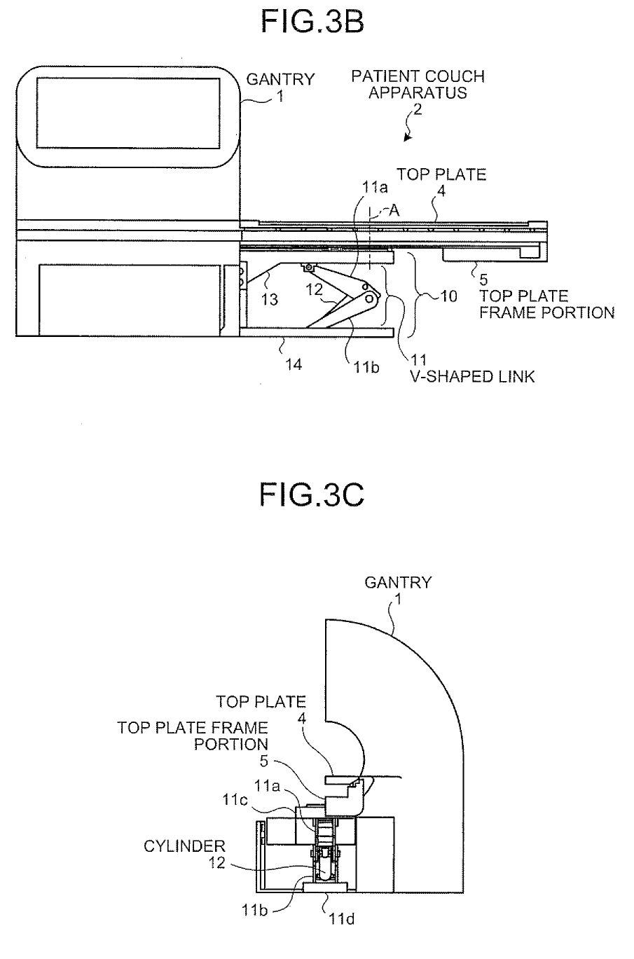

The up-down-lifting frame unit 10 supports the top plate frame portion 5 such that the top plate frame portion 5 is rotatable about the rotation axis A, and has the up-down lifting mechanism that moves the top plate frame portion 5 up-down directions. FIGS. 3A, 3B, and 3C are schematic diagrams illustrating an internal configuration of the MRI apparatus according to the first embodiment, in which FIG. 3A is a top view, FIG. 3B is a side view, and FIG. 3C is a front view.

As shown in FIGS. 3A to 3C, more specifically, the up-down-lifting frame unit 10 includes an upper support member 13 that supports the top plate frame portion 5 from below such that the top plate frame portion 5 is rotatable about the rotation axis A, and a lower support member 14 placed on a floor surface. The up-down-lifting frame unit 10 further has the V-shaped link 11 that includes two links; i.e., links 11a and 11b that cross each other in V-shape, and the cylinder 12 that serves as a driving device to change a crossing angle of the V-shaped link 11. The V-shaped link 11 and the cylinder 12 correspond to the up-down lifting mechanism.

The V-shaped link 11 and the cylinder 12 are constructed to change the crossing angle of the V-shaped link 11 so that the V-shaped link 11 and the cylinder 12 move the top plate frame portion 5 vertically via the upper support member 13. Both the V-shaped link 11 and the cylinder 12 are located closer to the gantry 1 than the rotation axis A of the top plate frame portion 5.

Because the V-shaped link 11 and the cylinder 12 are thus located closer to the gantry 1 than the rotation axis A of the top plate frame portion 5, a space serving as a foothold is secured under the top plate 4. Accordingly, an operator is allowed to put the operator's foot under the top plate 4 to secure an enough foothold for transfer of the subject P onto the top plate 4. Consequently, the transfer of the subject P is facilitated.

As shown in FIG. 3A, the upper support member 13 includes, between the upper support member 13 and the top plate frame portion 5, a rotary bearing 15 and a positioning unit 16. The rotary bearing 15 supports the top plate frame portion 5 such that the top plate frame portion 5 is rotatable about a predetermined rotation axis A. The positioning unit 16 is positioning means that positions the top plate frame portion 5 at a predetermined rotation angle. The top plate frame portion 5 can be rotated either manually or by providing a driving device, such as a motor, on the upper support member 13 to rotate the top plate frame portion 5 with the driving device.

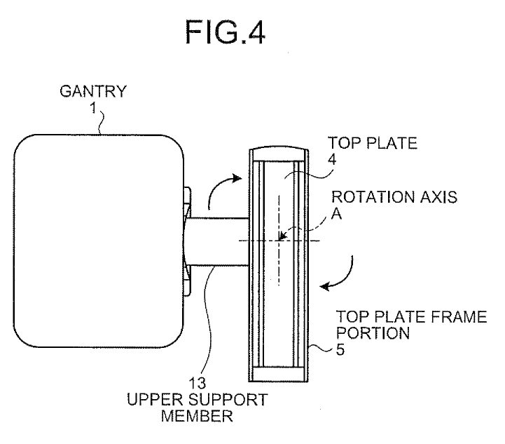

FIG. 4 is a diagram illustrating the top plate frame portion 5 in a rotated position. More specifically, as shown in FIG. 4, the upper support member 13 positions the top plate frame portion 5 at a rotation angle, clockwise about the rotation axis A, of approximately 90 degrees relative to a position at which the upper support member 13 is perpendicular to a front plane of the gantry 1. This position is referred to as a “subject-lying position”, at which the subject P is laid on the top plate 4. Meanwhile, the position (position of the top plate frame portion 5 shown in FIGS. 2A, 2B, and 2C), at which the top plate frame portion 5 is not rotated yet and the top plate 4 is perpendicular to the front plane of the gantry 1, is referred to as a “top plate reference position”.

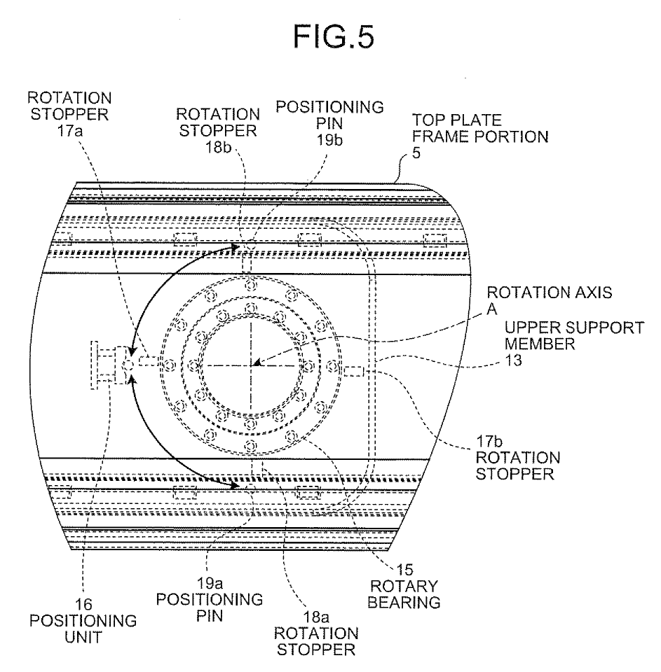

Subsequently, a rotating mechanism that rotates and positions the top plate frame portion 5 will be descried in detail. FIG. 5 is a diagram illustrating the rotating mechanism provided on the top plate frame portion 5 and the upper support member 13. FIG. 5 depicts structures of the upper support member 13 and the top plate frame portion 5 near the rotary bearing 15 shown in FIG. 3A.

A positioning unit 16 and rotation stoppers 17a and 17b shown in FIG. 5 are provided on an upper surface of the upper support member 13. Rotation stoppers 18a and 18b and positioning pins 19a and 19b are each provided on a lower surface of the top plate frame portion 5.

The positioning unit 16 provided on the upper support member 13 is engagement receiving means that retains the top plate frame portion 5 at a predetermined rotation angle. Each of the positioning pins 19a and 19b provided on the top plate frame portion 5 is engaging means that engages with the positioning unit 16.

More specifically, as shown in FIG. 5, the positioning unit 16 on the upper support member 13 and the positioning pins 19a and 19b on the top plate frame portion 5 are arranged concentrically about the rotation axis A. When the top plate frame portion 5 is rotated 90 degrees clockwise or counterclockwise, either one of the positioning pins 19a and 19b engages with the positioning unit 16, and hence the top plate frame portion 5 is positioned to the subject-lying position.

Thus, the positioning unit 16 and either one of the positioning pins 19a and 19b come into contact with each other, thereby positioning the top plate frame portion 5 to the subject-lying position. Therefore, it is possible to prevent the top plate frame portion 5 from unstably swinging when the subject P is laid on the top plate. Accordingly, an operator is allowed to lay the subject P on the top plate 4 easily with the top plate frame portion 5 at the rotated position.

The rotation stoppers 17a and 17b provided on the upper support member 13 and the rotation stoppers 18a and 18b provided on the top plate frame portion 5 are each engaging means that hold rotation of the top plate frame portion 5 at a predetermined rotation angle.

More specifically, as shown in FIG. 5, the rotation stoppers 17a and 17b, and 18a and 18b are arranged concentrically about the rotation axis A. When the top plate frame portion 5 is rotated 90 degrees clockwise or counterclockwise, the rotation stoppers 17a and 18a, and the rotation stoppers 18b and 18b engage each other, thereby holding rotation of the top plate frame portion 5. Hence, the rotation angle of the top plate frame portion 5 is limited so as not to exceed 90 degrees clockwise or counterclockwise from the top plate reference position.

In the above description, the top plate frame portion 5 is positioned to have a rotation angle of 90 degrees. Alternatively, positioning means; e.g., a hook or a pin, capable of holding the top plate frame portion 5 at any desired rotational angle can be provided on either one of the top plate frame portion 5 and the up-down-lifting frame unit 10 so that the top plate frame portion 5 is held at the desired rotation angle smaller than 90 degrees. According to this configuration, even when a direction, along which the subject P approaches the patient couch apparatus 2, is limited, the top plate frame portion 5 can be positioned to have the desired angle along the direction, thereby facilitating the lying of the subject P on the top plate 4.

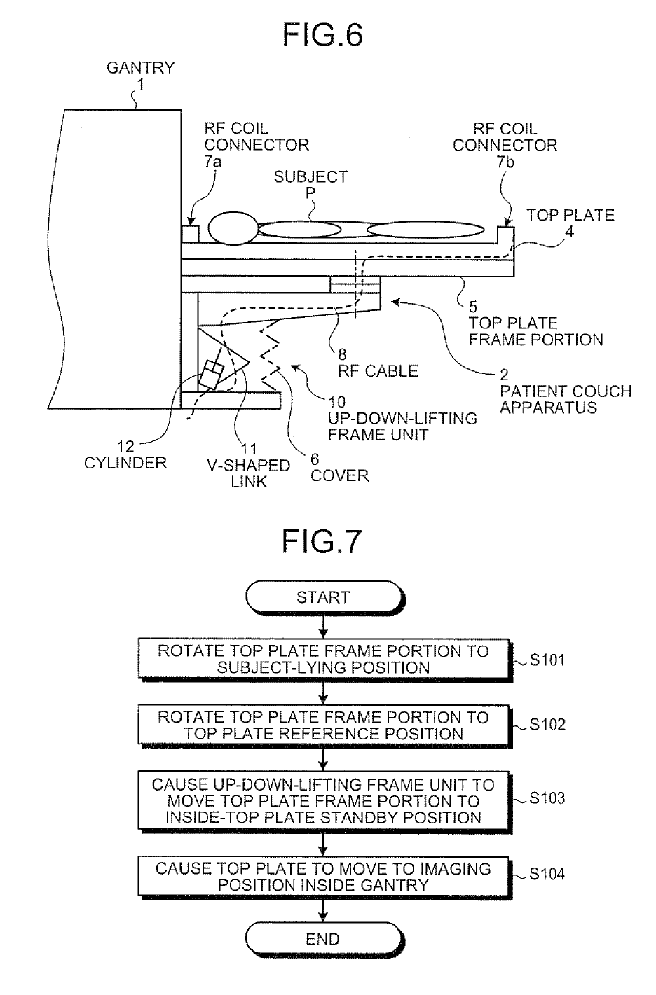

Meanwhile, a general patient couch apparatus used in an MRI apparatus includes, on its top plate, an RF coil connector to connect an RF coil attached to the subject P during imaging. The patient couch apparatus 2 according to the first embodiment also includes RF coil connectors at opposite ends of the top plate 4. FIG. 6 depicts the RF coil connectors provided on the patient couch apparatus 2 according the first embodiment. More specifically, as shown in FIG. 6, the patient couch apparatus 2 according to the first embodiment has, at the top plate reference position, an RF coil connector 7a at one end of the top plate frame portion 5 on a side of the gantry 1 and an RF coil connector 7b at the other end.

As shown in FIG. 6, in the patient couch apparatus 2 according to the first embodiment, an RF cable 8 that connects the RF coil connector 7b to an RF controller (not shown) is provided to extend through an inside of the top plate 4, an inside of the rotating mechanism, and an inside of the up-down-lifting frame unit 10 (inside of the cover 6 surrounding the V-shaped link 11).

This arrangement of the RF cable 8 allows, even when the top plate frame portion 5 is rotatably provided on the up-down-lifting frame unit 10, to situate the RF cable 8 without exposing the RF cable 8 to the outside of the MRI apparatus. Accordingly, the RF cable 8 is prevented from interfering the lying of the subject P on the top plate 4.

As described above, the rotation stoppers 17a, 17b, 18a, and 18b restrict the rotation of the top plate frame portion 5 so that the clockwise or counterclockwise rotational angle relative to the top plate reference position does not exceed 90 degrees. Accordingly, excessive twisting of the RF cable 8, which can lead to degradation or a break, is prevented.

Operations performed by the patient couch apparatus 2 according to the first embodiment for imaging will be described below. FIG. 7 is a flowchart of a sequence of the operations performed by the patient couch apparatus 2 according to the first embodiment for imaging. As shown in FIG. 7, when the MRI apparatus according to the first embodiment performs imaging of the subject P, the top plate frame portion 5 of the patient couch apparatus 2 first rotates to the subject-lying position, at which the subject P is laid on the top plate 4 (Step S101).

After the subject P has been laid on the top plate 4, the top plate frame portion 5 rotates from the subject-lying position to the top plate reference position, at which the top plate 4 is perpendicular to the front plane of the cavity 3 of the gantry 1 (Step S102).

Subsequently, the up-down-lifting frame unit 10 moves the top plate frame portion 5 from the top plate reference position to the inside-top plate standby position of a height, at which the top plate 4 can be inserted into the cavity 3 in the gantry 1 (Step S103).

The top plate 4 moves from the inside-top plate standby position to the imaging position inside the gantry 1 (Step S104). Imaging of the subject P is performed at the imaging position.

Because the top plate frame portion 5 thus rotates when the subject P is laid on the top plate 4, operability of the MRI apparatus during imaging can be improved.

The rotation of the top plate frame portion 5 and moving of the top plate 4 can be effected manually by an operator or automatically using a driving device, such as a motor, a controller, and the like.

The sequence of the operations pertaining to Steps S102 and S103 performed by the patient couch apparatus 2 can be interchanged. More specifically, according to the alternative sequence, after the subject P has been laid on the top plate 4, the up-down-lifting frame unit 10 moves the top plate frame portion 5 from the top plate reference position to the inside-top plate standby position (Step S103). Thereafter, the top plate frame portion 5 rotates to the top plate reference position (Step S102).

As described above, the MRI apparatus according to the first embodiment is configured as follows. The patient couch apparatus 2 includes the top plate 4 on which the subject P is laid, the top plate frame portion 5, and the up-down-lifting frame unit 10 that rotatably supports the top plate frame portion 5. The top plate frame portion 5 supports the top plate 4 such that the top plate 4 is movable into the imaging region in the cavity 3 of the gantry 1. The V-shaped link 11 and the cylinder 12 located closer to the gantry 1 than the rotation axis A of the top plate frame portion 5 allow the up-down-lifting frame unit 10 to move up and down. Accordingly, the subject P can be laid on the top plate 4 easily even when a direction, along which the subject P approaches the patient couch apparatus 2, is limited.

While the V-shaped link 11 and the cylinder 12 are used as the up-down lifting mechanism that moves the top plate frame portion 5 vertically in the description on the first embodiment, the present invention is not limited thereto, and the up-down lifting mechanism can be implemented using a lead screw that has a thread on an outer circumferential surface thereof. Another configuration that uses such a lead screw will be descried below as a second embodiment of the present invention.

For convenience of explanation, function units that have functions similar to those in the first embodiment with reference to the drawings will be denoted by like reference numerals, and descriptions thereof will be omitted.

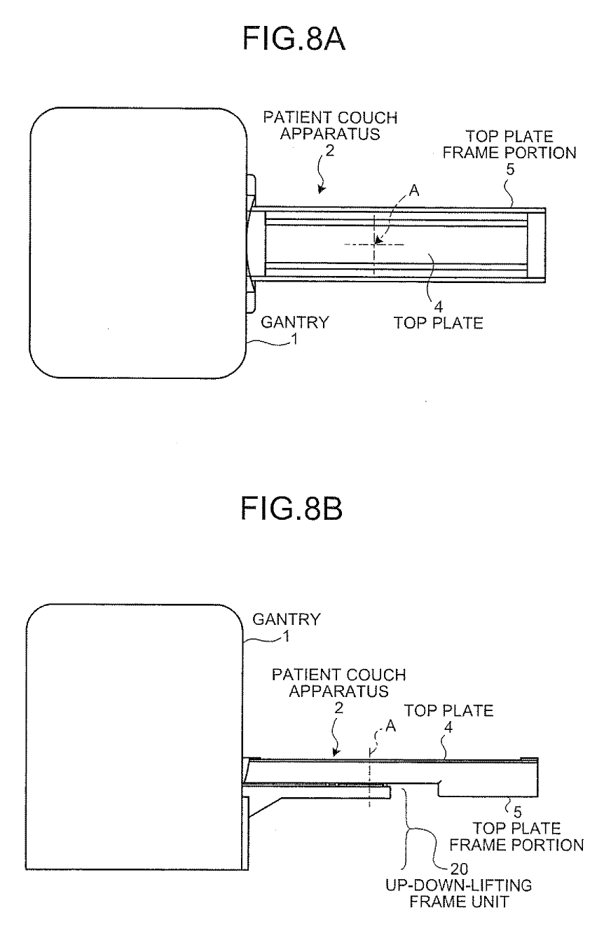

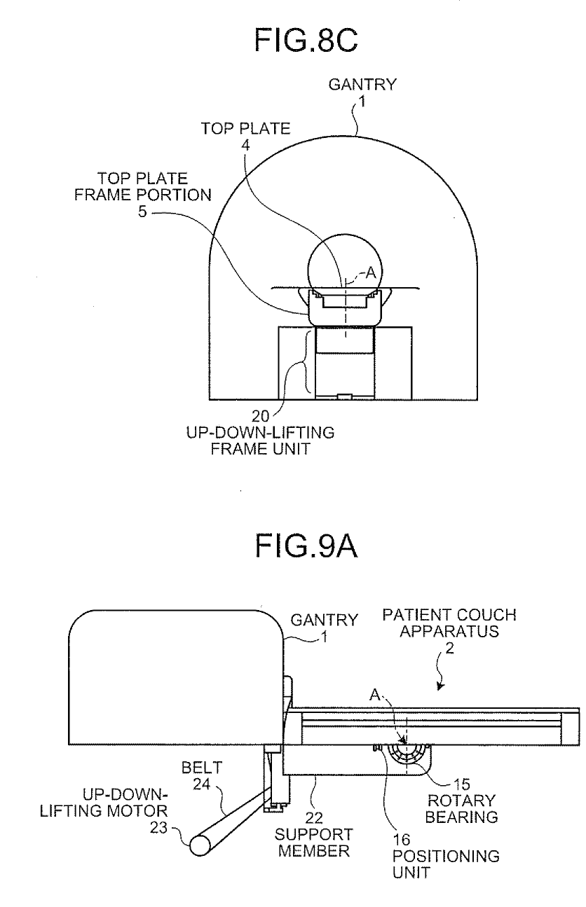

The overall configuration of an MRI apparatus according to the second embodiment will be described first. FIGS. 8A, 8B, and 8C are external views illustrating the overall configuration of the MRI apparatus according to the second embodiment, in which FIG. 8A is a top view, FIG. 8B is a side view, and FIG. 8C is a front view. As shown in FIGS. 8A, 8B, and 8C, the MRI apparatus according to the second embodiment includes the gantry 1 and the patient couch apparatus 2 as with the MRI apparatus according to the first embodiment.

As with the MRI apparatus according to the first embodiment, the patient couch apparatus 2 is located outside the gantry 1 and includes the top plate 4 on which the subject P is laid, the top plate frame portion 5, and an up-down-lifting frame unit 20.

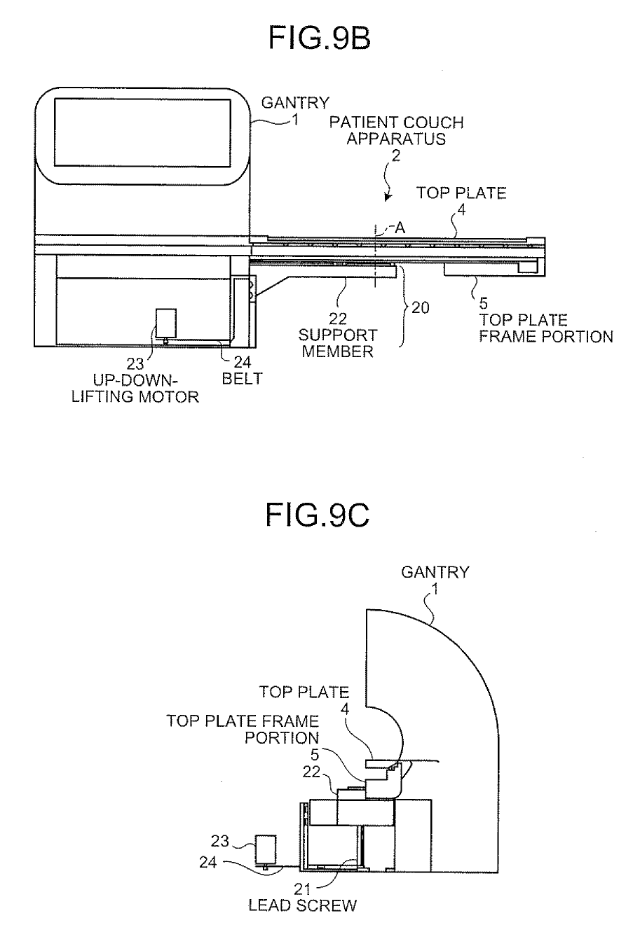

The up-down-lifting frame unit 20 supports the top plate frame portion 5 such that the top plate frame portion 5 is rotatable about the rotation axis A. The up-down-lifting frame unit 20 includes a lead screw 21 as the up-down lifting mechanism that moves the top plate frame portion 5 up-down directions. FIGS. 9A, 9B, and 9C are schematic diagrams illustrating an internal configuration of the MRI apparatus according to the second embodiment. FIG. 9A is a schematic diagram of the MRI apparatus as viewed from its top, FIG. 9B is a schematic diagram of the same as viewed from a side, and FIG. 9C is a schematic diagram of the same as viewed from its front.

As shown in FIGS. 9A, 9B, and 9C, more specifically, the up-down-lifting frame unit 20 includes a support member 22 that supports the top plate frame portion 5 from below such that the top plate frame portion 5 is rotatable about a predetermined rotation axis A. The up-down-lifting frame unit 20 further includes the lead screw 21 that supports the support member 22 from below, and an up-down-lifting motor 23 and a belt 24 that rotate the lead screw 21.

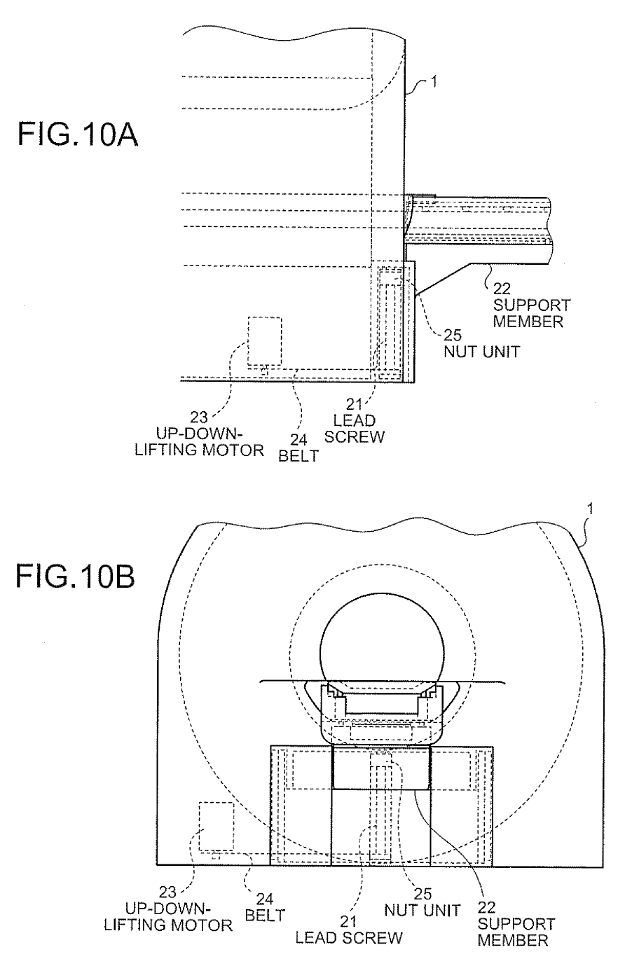

FIGS. 10A and 10B are diagrams illustrating the up-down lifting mechanism of the up-down lifting frame 20 according to the second embodiment. As shown in FIGS. 10A and 10B, the lead screw 21 is provided upright at a position closer to the gantry 1 than the rotation axis A of the top plate frame portion 5. As shown in FIGS. 10A and 10B, the support member 22 has, at an end thereof on a side of the gantry 1, a nut unit 25 that has screw channels therein to receive the thread of the lead screw 21. As shown in FIGS. 10A and 10B, the up-down-lifting motor 23 and the belt 24 drive the lead screw 21 to rotate with the lead screw 21 screwed into the nut unit of the support member 22, thereby enabling to move the support member 22 up-down directions.

Because the lead screw 21 is thus located closer to the gantry 1 than the rotation axis A of the top plate frame portion 5, a space for a foothold is secured under the top plate 4. This arrangement allows, when the subject P is laid on the top plate 4 by an operator, to put the operator's foot under the top plate 4 to secure an enough foothold, thereby facilitating the lying of the subject P.

As described above, the MRI apparatus according to the second embodiment is configured as follows. The patient couch apparatus 2 includes the top plate 4 on which the subject P is laid, the top plate frame portion 5, and the up-down-lifting frame unit 20. The top plate frame portion 5 supports the top plate 4 such that the top plate 4 is movable into the imaging region in the cavity 3 of the gantry 1. The lead screw 21 located closer to the gantry 1 than the rotation axis A of the top plate frame portion 5 allows the up-down-lifting frame unit 20 to move up and down. Accordingly, the subject P can be laid on the top plate 4 easily even when a direction, along which the subject P approaches the patient couch apparatus 2, is limited.

In the descriptions on the first and second embodiments, the top plate frame portion 5 supports the top plate 4 such that the top plate 4 is movable into the imaging region in the cavity 3 of the gantry 1. However, alternatively, the top plate 4 included in the patient couch apparatus 2 can be detachably constructed so that the top plate 4 can be used as a portion of a stretcher that carries the subject P.

Such a configuration, in which the top plate 4 is detachable to be used as a portion of a stretcher, will be descried below as a third embodiment of the present invention. Meanwhile, the third embodiment will be described based on the MRI apparatus according to the second embodiment, and function units that have functions similar to those having already been referred to will be denoted by like reference numerals and descriptions thereof will be omitted.

In the MRI apparatus according to the third embodiment, the patient couch apparatus 2 includes the top plate 4 that is detachably supported by the top plate frame portion 5. After being removed from the top plate frame portion 5, the top plate 4 is attached to a trolley on casters to be used as a portion of the stretcher 30 that carries the subject P. Transfer of the top plate 4 between the patient couch apparatus 2 and the stretcher 30 of the MRI apparatus according to the third embodiment will be described below.

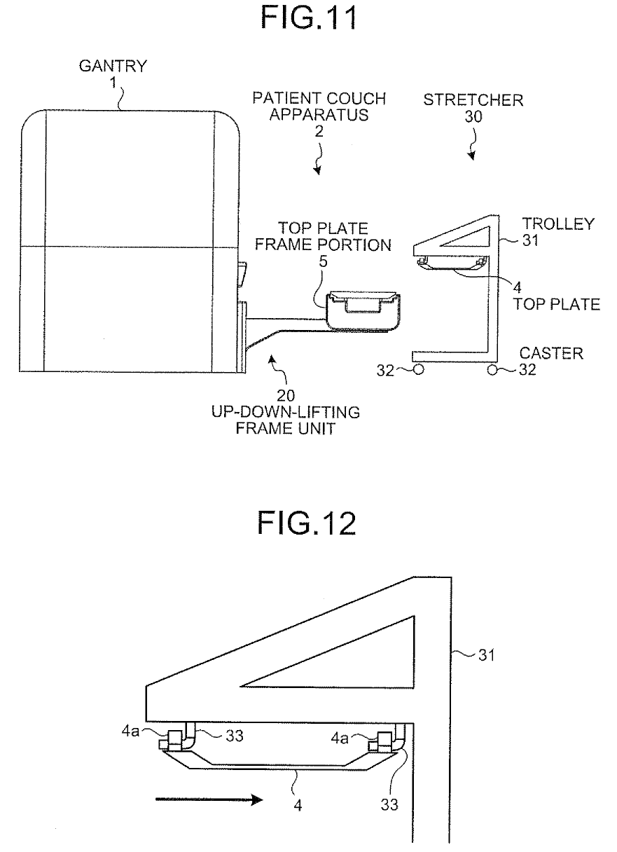

First, transfer of the top plate 4 from the stretcher 30 to the patient couch apparatus 2 will be described. FIGS. 11 to 15 are first to fifth diagrams of the transfer of the top plate 4 from the stretcher 30 to the patient couch apparatus 2 of the MRI apparatus according to the third embodiment. FIGS. 11 to 15 depict the patient couch apparatus 2 with the top plate frame portion 5 having already been positioned to have a rotation angle of approximately 90 degrees.

FIG. 11 depicts configurations of the MRI apparatus and the stretcher 30 according to the third embodiment. As shown in FIG. 11, the stretcher 30 is constructed by removing the top plate 4 from the patient couch apparatus 2 and attaching the top plate 4 to a trolley 31 on casters 32.

FIG. 12 is a schematic diagram for explaining the top plate 4 being attached to the trolley 31, depicting portions, at which the top plate 4 is attached to the trolley 31. As shown in FIG. 12, a predetermined number of hooks 33 are provided on the trolley 31 of the stretcher 30 at locations at which the top plate 4 is attached to the trolley 31. Fittings 4a to be received by the hooks 33 on the trolley 31 into hook engagement are provided on an upper portion of the top plate 4 in equal number to the hooks 33. Moving the top plate 4 in a direction indicated by the arrow in FIG. 12 brings the fittings 4a into hook engagement with the hooks 33, thereby attaching the top plate 4 to the trolley 31. In contrast, moving the top plate 4 in a direction opposite from that indicated by the arrow in FIG. 12 detaches the top plate 4 from the trolley 31.

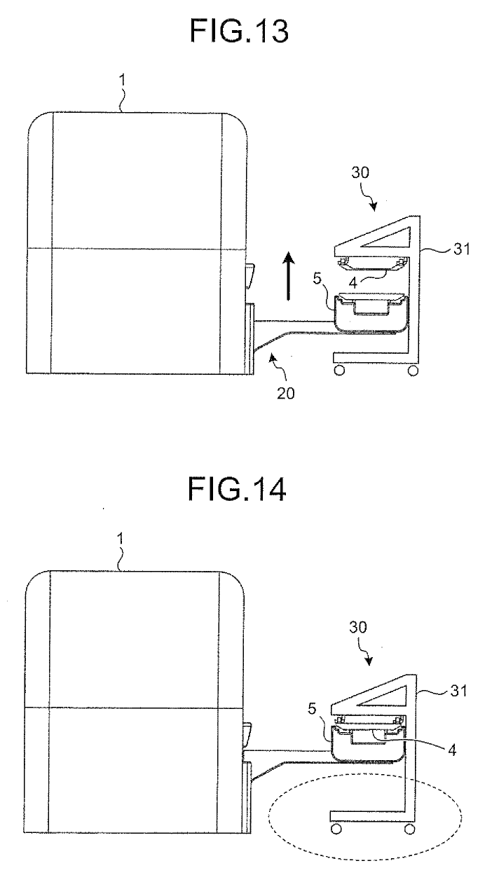

FIG. 13 depicts a state where the stretcher 30 is moved until the top plate 4 positions immediately above the top plate frame portion 5. When the top plate frame portion 5 is moved upward by the up-down-lifting frame unit 20 from this state, the top plate 4 attached to the trolley 31 is mounted on an upper portion of the top plate frame portion 5 as shown in FIG. 14.

As with the first and second embodiments, the MRI apparatus according to the third embodiment allows to secure a space (space indicated by a dashed ellipse in FIG. 14) below the top plate frame portion 5. Accordingly, by inserting a leg portion of the stretcher 30 into the space, the stretcher 30 can be situated alongside the patient couch apparatus 2.

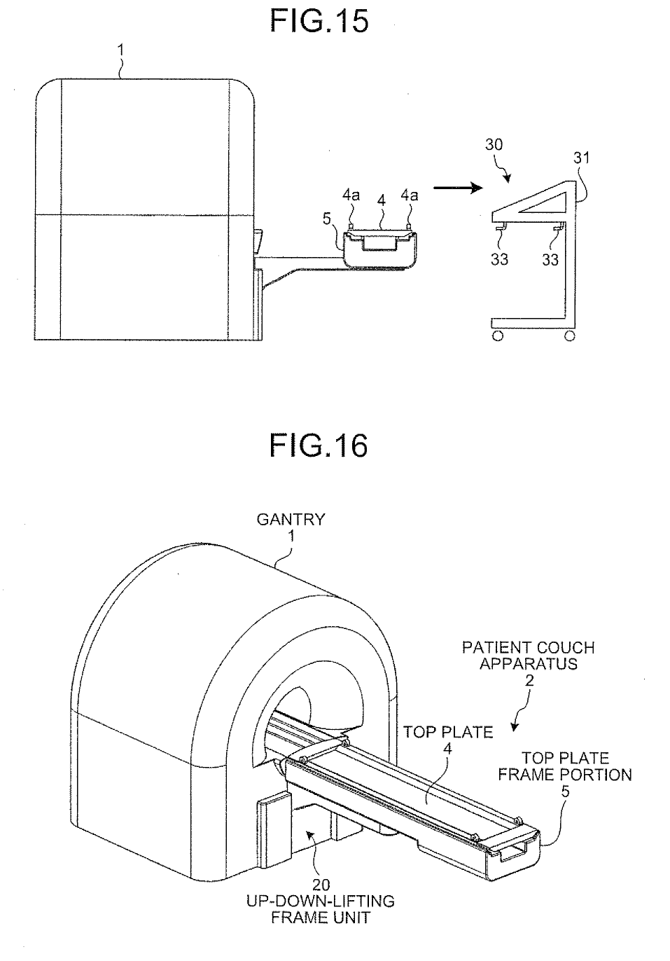

When the stretcher 30 is moved away from the top plate frame portion 5 as shown in FIG. 15, the fittings 4a are detached from the hooks 33 on the trolley 31. Consequently, the top plate 4 is left on the upper portion of the top plate frame portion 5.

As described above, in the MRI apparatus according to the third embodiment, the top plate 4 is detachably provided on the top plate frame portion 5. Accordingly, the top plate 4 having been attached to the trolley 31 can be removed from the trolley 31 and attached to the patient couch apparatus 2. Hence, the MRI apparatus according to the third embodiment allows to transfer the subject P carried on the stretcher 30 onto the patient couch apparatus 2 with the top plate 4 in an integrated manner, thereby further facilitating the transfer of the subject P onto the patient couch apparatus 2.

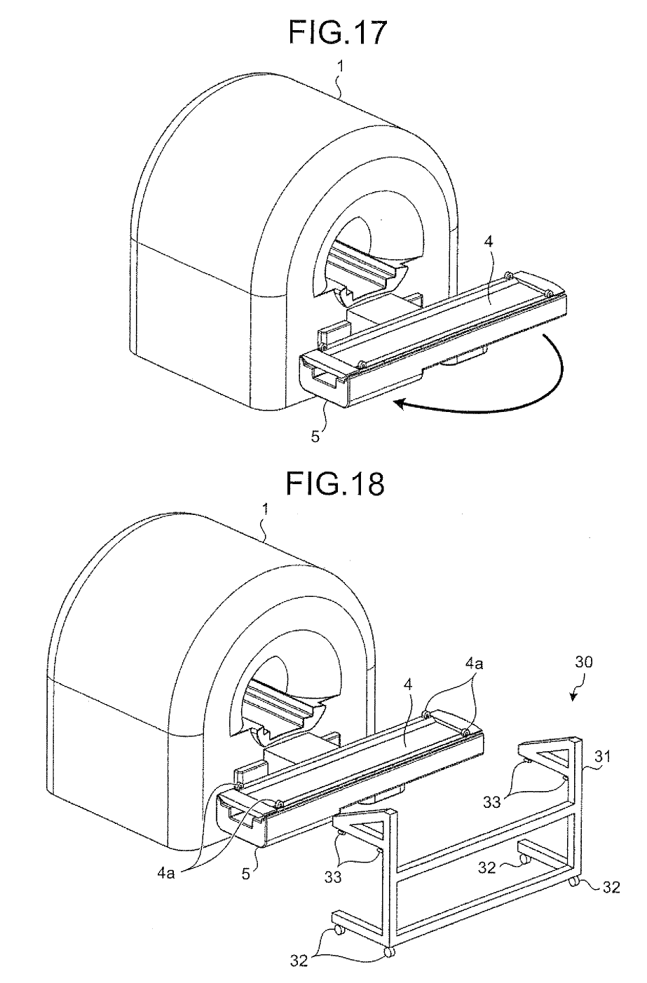

Transfer of the top plate 4 from the patient couch apparatus 2 to the stretcher 30 will be described below. FIGS. 16 to 21 are first to sixth diagrams illustrating the transfer of the top plate 4 to the stretcher 30 from the patient couch apparatus 2 of the MRI apparatus according to the third embodiment. FIG. 16 depicts the MRI apparatus of a state where the top plate frame portion 5 is not rotated yet. From the state shown in FIG. 16, the top plate frame portion 5 is rotated as shown in FIG. 17.

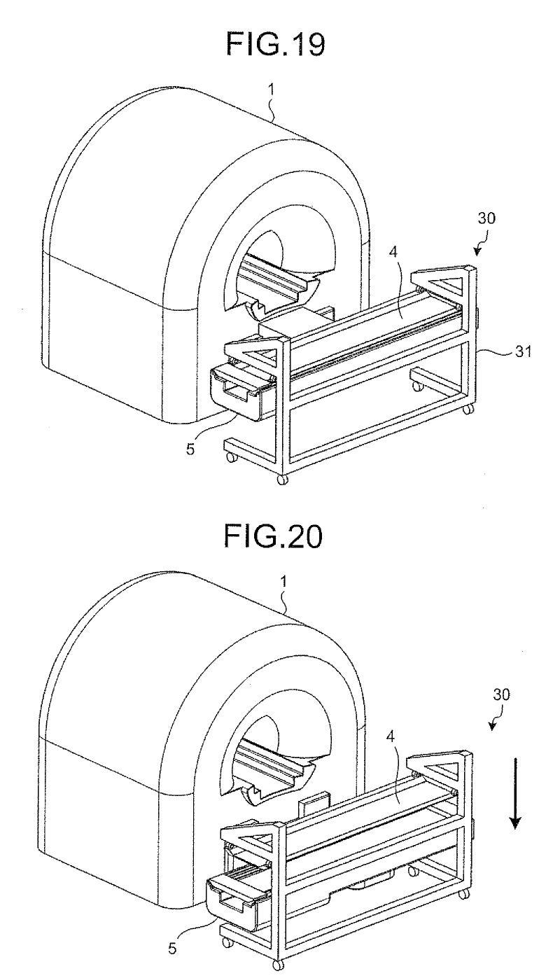

Subsequently, the stretcher 30 is set to an orientation and position from which the stretcher 30 can approach the MRI apparatus as shown in FIG. 18. In this state, the top plate frame portion 5 has already been moved upward to bring the fittings 4a on the top plate 4 at a same height as the hooks 33 on the trolley 31. From this state, as shown in FIG. 19, the stretcher 30 is moved to overlap the top plate frame portion 5. Consequently, the fittings 4a come into hook engagement with the hooks 33 on the trolley 31, and hence the top plate 4 is attached to the trolley 31.

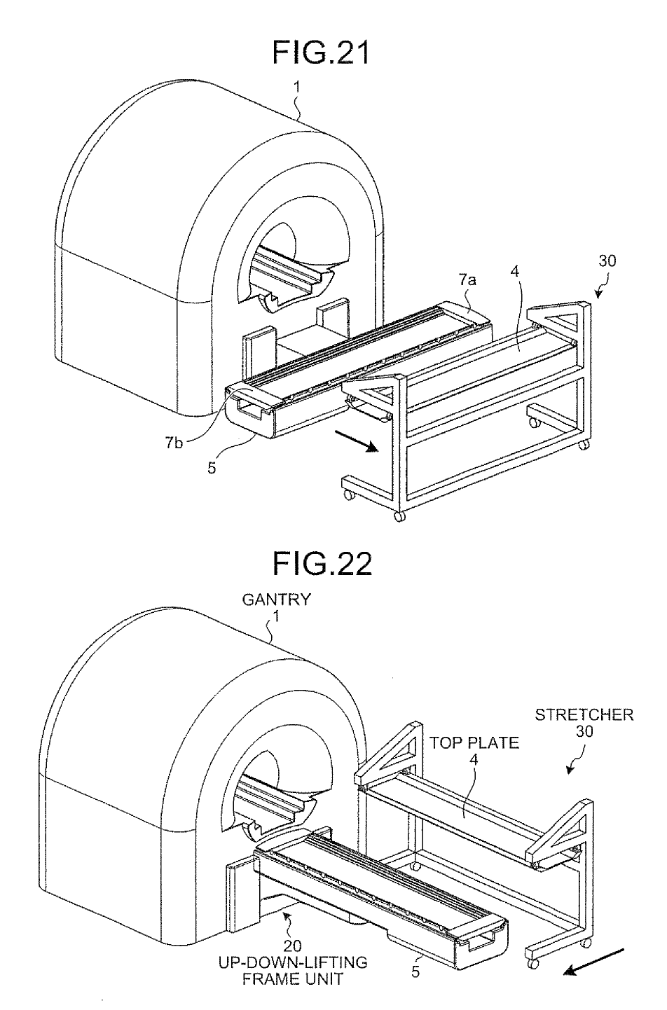

When the top plate frame portion 5 is moved downward by the up-down-lifting frame unit 20 from this state, the top plate 4 is detached from the upper portion of the top plate frame portion 5 as shown in FIG. 20. When, as shown in FIG. 21, the stretcher 30 is moved away from the top plate frame portion 5, the top plate 4 is also moved with the stretcher 30 in an integrated manner.

As shown in FIG. 21, in the MRI apparatus according to the third embodiment, the RF coil connectors 7a and 7b are provided at ends of the top plate frame portion 5 so as to be left on the patient couch apparatus 2 even after the top plate 4 is removed.

As described above, in the MRI apparatus according to the third embodiment, the top plate 4 is detachably provided on the top plate frame portion 5. Accordingly, the top plate 4 attached to the patient couch apparatus 2 can be removed from the patient couch apparatus 2 and attached to the stretcher 30. Therefore, in the MRI apparatus according to the third embodiment, the subject P laid on the patient couch apparatus 2 along with the top plate 4 can be removed to the stretcher 30. Hence, the transfer of the subject P to the patient couch apparatus 2 is further facilitated.

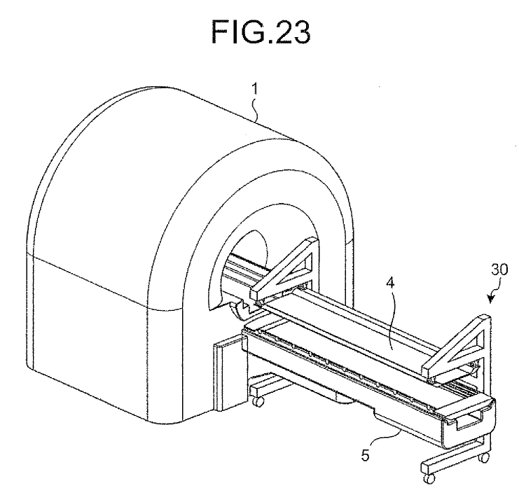

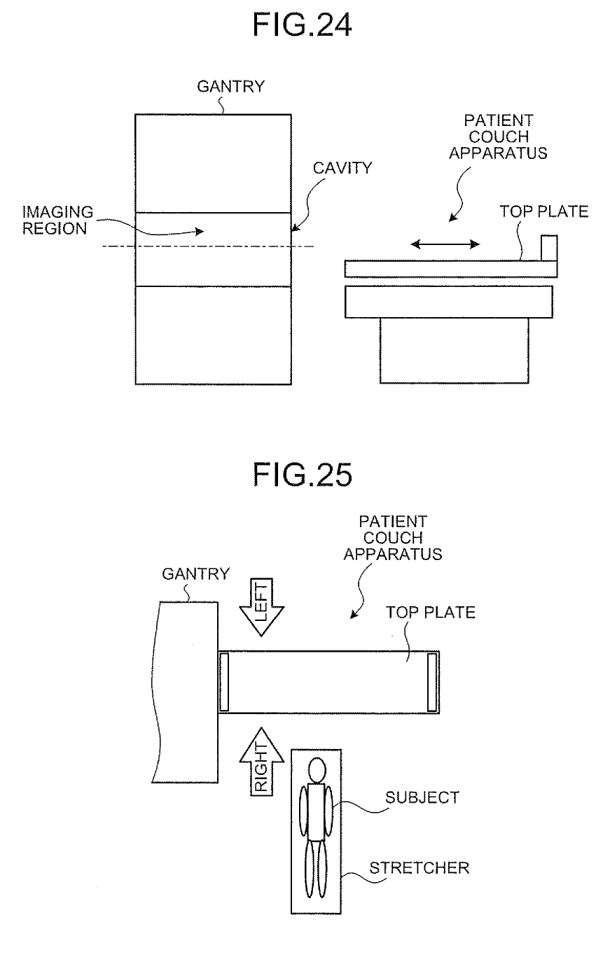

In the above description, the top plate 4 is transferred to and from the stretcher 30 in the state where the top plate frame portion 5 is rotated. However, in the MRI apparatus according to the third embodiment, the top plate 4 can be moved similarly even when the top plate frame portion 5 is not rotated. FIGS. 22 and 23 are first and second diagrams illustrating the transfer of the top plate 4 in the MRI apparatus according to the third embodiment, the transfer being effected without rotating the top plate frame portion 5.

More specifically, as shown in FIG. 22, the MRI apparatus according to the third embodiment allows, as with the first and second embodiments, to secure a space below the top plate frame portion 5 even when the top plate frame portion 5 is not rotated. Thus, in the MRI apparatus according to the third embodiment, because the stretcher 30 can be situated alongside the patient couch apparatus 2 by inserting the leg portion of the stretcher 30 into the space secured under the top plate frame portion 5 even when the top plate frame portion 5 is not rotated, the top plate 4 can be transferred between the stretcher 30 and the patient couch apparatus 2 as shown in FIG. 23.

While the embodiments have described applications of the present invention to patient couch apparatuses used in an MRI apparatus, application of the invention is not limited thereto, and the invention can be similarly applied to patient couch apparatuses for use in other diagnostic imaging apparatuses, such as an X-ray CT apparatus.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A patient couch apparatus comprising:
a top plate on which a subject is laid; and
a frame portion that is provided on a gantry that performs imaging the subject, movable into an imaging region inside the gantry, and rotatably supports the top plate in an area outside the imaging region.

2. The apparatus according to claim 1, wherein the top plate is detachably supported by the frame portion and is attachable to a trolley on a stretcher that carries the subject.

3. The apparatus according to claim 1, wherein the frame portion includes
a first frame portion that supports the top plate movably into the imaging region, and
a second frame portion that rotatably supports the first frame portion, wherein
the first frame portion has positioning-and-engaging units arranged concentrically about a rotation axis of the top plate; and
the second frame portion has an engagement receiving unit between the first frame portion and the second frame portion, the engagement receiving unit being positioned by engaging with one of the positioning-and-engaging units at a predetermined rotation angle.

4. The apparatus according to claim 3, wherein the top plate is detachably supported by the frame portion and is attachable to a trolley on a stretcher that carries the subject.

5. The apparatus according to claim 3, wherein the engagement receiving unit is positioned to have a rotation angle of approximately 90 degrees.

6. The apparatus according to claim 5, wherein the top plate is detachably supported by the frame portion and is attachable to a trolley on a stretcher that carries the subject.

7. A patient couch apparatus comprising:
a top plate on which a subject is laid;
an up-down lifting mechanism that is provided on a gantry that performs imaging the subject, and supports the top plate movably to up-down directions.

8. The apparatus according to claim 7, wherein
the up-down lifting mechanism supports the top plate via a top plate frame portion; and
the top plate is detachably supported by the top plate frame portion and is attachable to a trolley on a stretcher that carries the subject.

9. The apparatus according to claim 7, wherein the up-down lifting mechanism includes a V-shaped link that supports the top plate and a driving device that changes a crossing angle of the V-shaped link.

10. The apparatus according to claim 9, wherein
the up-down lifting mechanism supports the top plate via a top plate frame portion; and
the top plate is detachably supported by the top plate frame portion and is attachable to a trolley on a stretcher that carries the subject.

11. The apparatus according to claim 7, wherein the up-down lifting mechanism includes
a support member that supports the top plate;
a lead screw that is screwed into a nut unit provided in the support member; and
a driving device that drives to rotate the lead screw.

12. The apparatus according to claim 11, wherein
the up-down lifting mechanism supports the top plate via a top plate frame portion; and
the top plate is detachably supported by the top plate frame portion and is attachable to a trolley on a stretcher that carries the subject.

13. A diagnostic imaging apparatus comprising:
a gantry that performs imaging a subject; and
a patient couch apparatus on which the subject is laid, wherein the patient couch apparatus includes
a top plate on which the subject is laid; and
a frame portion that is provided on the gantry, supports the top plate movably into an imaging region inside the gantry, and rotatably supports the top plate in an area outside the imaging region.

14. A diagnostic imaging apparatus comprising:
a gantry that performs imaging a subject; and
a patient couch apparatus on which the subject is laid, wherein the patient couch apparatus includes
a top plate on which the subject is laid; and
an up-down lifting mechanism that is provided on the gantry and supports the top plate movably to up-down directions.

15. A method of operating a patient couch apparatus comprising:
rotating a top plate frame portion that supports a top plate on which a subject is laid, to a predetermined subject-lying position at which the subject is laid on the top plate;
rotating the top plate frame portion from the subject-lying position to a predetermined top plate reference position at which the top plate is perpendicular to a front plane of a cavity of a gantry that performs imaging the subject;
moving the top plate frame portion from the top plate reference position to an inside-top plate standby position of a height at which the top plate is allowed to be inserted into the cavity of the gantry, by using an up-down lifting mechanism that is provided on the gantry and supports the top plate frame portion movably to up-down directions; and
moving the top plate from the inside-top plate standby position to a predetermined imaging position inside the gantry.
